(19) United States
(12) Patent Application Publication
(10) Pub. No.: US 2025/0195786 A1
(43) Pub. Date: Jun. 19, 2025

(54) DEVICES FOR USE WITH A SYRINGE

(71) Applicant: Boston Scientific Scimed, Inc., Maple Grove, MN (US)

(72) Inventors: Ajay Gupta, Shoreview, MN (US); Randy Raymond, Maple Grove, MN (US); Mark Steven Smith, Coon Rapids, MN (US)

(73) Assignee: Boston Scientific Scimed, Inc., Maple Grove, MN (US)

(21) Appl. No.: 18/980,288

(22) Filed: Dec. 13, 2024

Related U.S. Application Data
(60) Provisional application No. 63/609,986, filed on Dec. 14, 2023.

(51) Int. Cl. A61M 5/315

(52) U.S. Cl. CPC A61M 5/31586

(57) ABSTRACT

Devices and/or adapters for use with a syringe are disclosed. An example device for use with a syringe may include a first member having a holding region configured to engage a flange of a syringe barrel. The device may also include a second member coupled to the first member. The second member may have an end surface configured to engage a syringe plunger. Relative movement between the first member and the second member may shift the syringe plunger within the syringe barrel.

### CROSS-REFERENCE TO RELATED APPLICATIONS

The application claims the benefit of U.S. Provisional Patent Application Ser. No. 63/609,986 filed on Dec. 14, 2023, the disclosure of which is incorporated herein by reference.

### TECHNICAL FIELD

The present disclosure pertains to medical devices, and methods for manufacturing medical devices. More particularly, the present disclosure pertains to devices for use with a syringe.

### BACKGROUND

A wide variety of medical devices have been developed for medical use, for example, intravascular use. Some of these devices include guidewires, catheters, syringes, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A device for use with a syringe is disclosed. The device comprises: a first member having a holding region configured to engage a flange of a syringe barrel; a second member coupled to the first member, the second member having an end surface configured to engage a syringe plunger head; and wherein relative movement between the first member and the second member shifts the syringe plunger within the syringe barrel.

Alternatively or additionally to any of the embodiments above, the first member includes a substantially cylindrical body.

Alternatively or additionally to any of the embodiments above, the holding region includes a socket configured to secure the flange to the first member.

Alternatively or additionally to any of the embodiments above, a helical groove is formed along an interior region of the cylindrical body.

Alternatively or additionally to any of the embodiments above, the second member includes an actuator having an external thread, the external thread being configured to engage the helical groove.

Alternatively or additionally to any of the embodiments above, the second member is rotatable relative to the first member.

Alternatively or additionally to any of the embodiments above, the holding region includes a helical groove formed along an interior region of the cylindrical body.

Alternatively or additionally to any of the embodiments above, the cylindrical body is rotatable relative to the flange, and wherein rotation of the cylindrical body translates the flange relative to the syringe plunger.

Alternatively or additionally to any of the embodiments above, the first member includes a lower handle having one or more finger holding regions.

Alternatively or additionally to any of the embodiments above, the second member includes an upper push handle.

Alternatively or additionally to any of the embodiments above, further comprising one or more alignment members extending between the first member and the second member.

An adapter for use with a syringe is disclosed. The adapter comprises: a substantially cylindrical body having a holding region configured to engage a flange of a syringe barrel; a push member coupled to the substantially cylindrical body, the push member having an end surface configured to engage a syringe plunger; and wherein relative movement between the substantially cylindrical body and the push member shifts the syringe plunger within the syringe barrel.

Alternatively or additionally to any of the embodiments above, the holding region includes a socket configured to secure the flange to the substantially cylindrical body.

Alternatively or additionally to any of the embodiments above, a helical groove is formed along an interior region of the substantially cylindrical body.

Alternatively or additionally to any of the embodiments above, the push member includes an external thread, the external thread being configured to engage the helical groove.

Alternatively or additionally to any of the embodiments above, the push member is rotatable relative to the substantially cylindrical body.

Alternatively or additionally to any of the embodiments above, the holding region includes a helical groove formed along an interior region of the cylindrical body.

Alternatively or additionally to any of the embodiments above, the substantially cylindrical body is rotatable relative to the flange, and wherein rotation of the substantially cylindrical body translates the flange relative to the syringe plunger.

An adapter for use with a syringe is disclosed. The adapter comprises: a lower handle having one or more finger regions and a socket configured to engage a flange of a syringe barrel; an upper push handle coupled to the lower handle, the upper push handle having an end surface configured to engage a syringe plunger; and wherein relative movement between the lower handle and the upper push handle shifts the syringe plunger within the syringe barrel.

Alternatively or additionally to any of the embodiments above, further comprising one or more alignment members extending between the lower handle and the upper push member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic overview of an example syringe being used to infuse an embolic material into a body lumen.

FIG. 2 is a perspective view of an example syringe.

FIGS. 3-4 illustrate an example device for use with a syringe.

FIGS. 5-7 illustrate an example device for use with a syringe.

FIGS. 8-9 illustrate an example device for use with a syringe.

FIGS. 10-12 illustrate an example device for use with a syringe.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

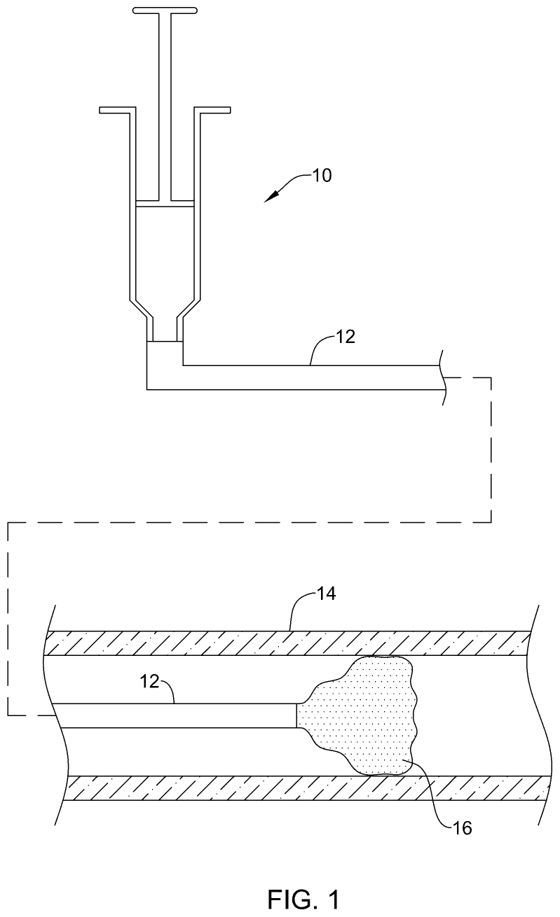

FIG. 1 schematically illustrates a syringe 10 coupled to a catheter or tubular member 12. The tubular member 12 may be disposed within a body lumen or blood vessel 14. The syringe 10 and tubular member 12 may be used to deliver an embolic material 16 within the blood vessel 14. In some instances, the embolic material 16 may be delivered to the blood vessel 14 in order to embolize the blood vessel 14 and/or stop bleeding. This is one example of a clinical intervention where a syringe 10 may be used to deliver a liquid substance (e.g., the embolic material 16). It can be appreciated that a number of different interventions and/or systems may be used in conjunction with a syringe (e.g., the syringe 10) at a number of different body locations (e.g., blood vessels, body cavities, body lumens, ducts, passageways, etc.) and to deliver a number of different materials. The syringe 10 and the intervention schematically depicted in FIG. 1 is not intended to be limiting as a number of different clinical interventions are contemplated.

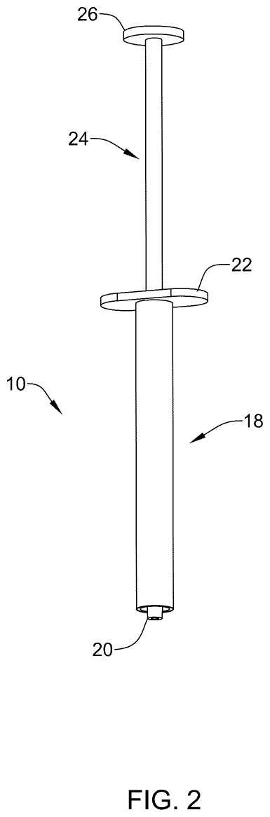

The syringe 10 is shown in FIG. 2. Again, the syringe 10 depicted in FIG. 2 is meant to be just one example of a syringe. A variety of different syringes and/or fluid delivery devices are contemplated. In the example depicted in FIG. 2, it can be seen that the syringe 10 may include a syringe barrel 18. A connector 20 may be disposed at an outflow end of the syringe 10. A flange or flanged region 22 may be disposed at an opposite end of the syringe barrel 18. A plunger 24 may be slidably disposed within the syringe barrel 18. The plunger 24 may include a plunger head or head region 26. A clinician may use the syringe 10 to deliver a fluid by pressing the plunger head 26 while holding the flange 22 so that the plunger 24 shifts within the syringe barrel 18. Movement of the plunger 24 urges the fluid out of the syringe barrel 18.

A number of clinical interventions include the delivery of a liquid substance to the patient using a syringe. In some cases the liquid may be viscous. For example, some liquid embolic materials may be viscous and could require a substantial force to overcome backpressure in order to deliver the substance. For example, liquid embolic materials such as OBSIDIO™ Liquid Embolic material (commercially available from Boston Scientific) may be viscous and could require increased forces/pressure in order to pass the material through a syringe and/or catheter. Disclosed herein are devices that may help to manage forces associated with delivery of liquid substances, for example via a syringe. This may improve the deliverability of viscous substances to a patient during an intervention.

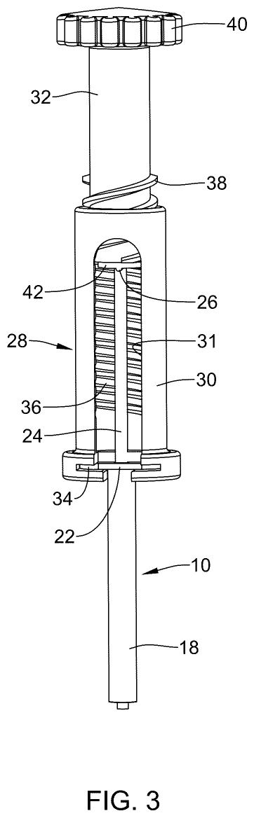

FIG. 3 illustrates an example device 28 for use with a syringe (e.g., the syringe 10). In some instances, the device 28 may be termed an adapter or a syringe adapter. In general, the device 28 is designed to improve the ability of a user to deliver a liquid substance with the syringe 10. This is because, for example, the device 28 may make it easier for a clinician to apply forces sufficient to overcome backpressure when delivering a fluid (e.g., a viscous fluid such as a liquid embolic) with the syringe 10. Thus, the device 28 may be used in conjunction with a syringe (e.g., the syringe 10 or similar) in order to improve the ability of a user to deliver a substance with the syringe.

The device 28 may include a first part or member 30 and a second part or member 32 coupled to the first member 30. The first member 30 may include a holding region or socket 34. In general, the holding region 34 may be configured to engage the flange 22 of the syringe 10 and/or secure the flange 22 to and/or relative to the first member 30. For example, the holding region 34 may be formed as an opening in the first member 30 that allows the flange 22 to be inserted therein. When doing so, the flange 22 may be secured (e.g., releasably secured) to the first member 30. In FIG. 3, the first member 30 is shown with a side opening that provides access to the holding region 34. Thus, the flange 22 may be inserted into the holding region 34 via lateral access via the side opening.

The first member 30 may also include a body (e.g., a generally cylindrical body) having an internal thread or helical groove 36 formed along an interior region thereof. The helical groove 36 may extend along substantially the full length of the first member 30. Alternatively, the helical groove 36 may extend along a portion or multiple portions of the first member 30. In FIG. 3, the first member 30 is shown partially cutaway so that the helical groove 36 visible. The cutaway portion, however, may also be present in the first member 30 (e.g., the cutaway portion, bearing reference number 31 in FIG. 3, may be a structural feature of the first member 30), which may make it easier for a user to visualize the helical groove 36 and/or the position of the plunger 24 within the first member 30.

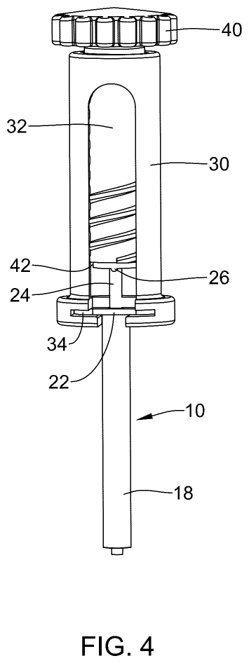

The second member 32 may resemble a piston or rod that is movable within the first member 30. In at least some instances, the second member 32 may include an external thread or threaded region 38. In general, the external thread 38 is configured to engage the helical groove 36 of the first member 30. The second member 32 may also include a knob or actuator 40. The second member 32 may also include a head or end surface 42 configured to engage the syringe plunger head 26. In use, the actuator 40 may be used to rotate the second member 32 relative to the first member 30. When doing so, the second member 32 may shift relative to the first member 30 as shown in FIG. 4. The engagement between the end surface 42 and the syringe plunger head 26 may cause the plunger 24 to shift within the syringe barrel 18.

It can be appreciated that in FIGS. 3-4, the helical groove 36 is depicted as being disposed along the first member 30 and the external thread 38 is depicted as being disposed along the second member 32. This is not intended to be limiting. Arrangements are contemplated where the first member 30 may include a thread or projection (e.g., akin to a male thread) disposed along an interior region thereof and the second member 32 may have a helical groove or thread (e.g., akin to a female thread) thereon that is configured to engage the male thread of the first member 30. In other words, the arrangement of the groove(s) and/or thread(s) for the various members of the devices disclosed herein may be arranged in a suitable manner and, in some cases, can be reversed for what is shown/described.

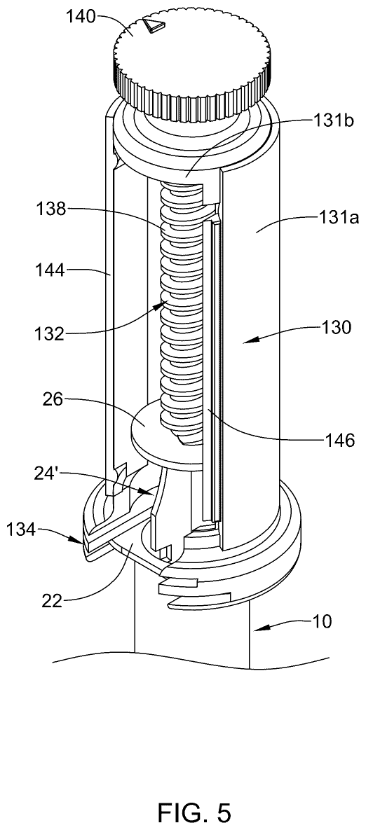

FIGS. 5-7 illustrate another example device 128 that may be similar in form and function to other devices disclosed herein. The device 128 may include a first member 130 and a second member 132 coupled to the first member 130. The first member 130 may include a holding region or socket 134. In general, the holding region 134 may be configured to engage the flange 22 of the syringe 10 and/or secure the flange 22 to the first member 130. It can be seen in FIG. 5 that a slightly varied plunger 24′ is depicted for use with the syringe 10. This is not intended to be limiting and, instead, is meant to be exemplary of the notion that a variety of different syringes exist and many of these syringes can be used with the device 128 and/or other devices disclosed herein.

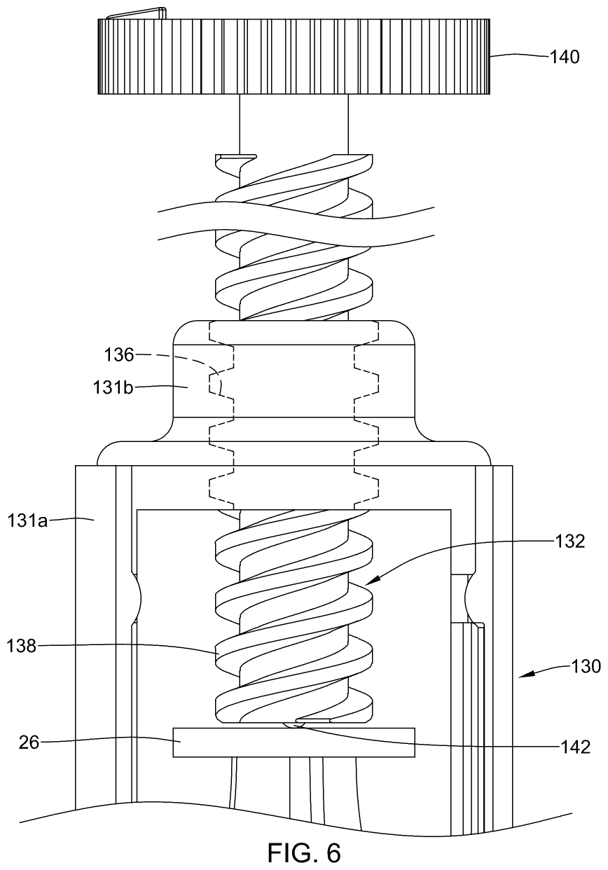

The first member 130 may include a body 131a (e.g., a generally cylindrical body) and a threaded region 131b having an internal thread or helical groove 136 formed along an interior region thereof (e.g., the helical groove 136 is shown in FIG. 6). In some instances, the threaded region 131b may be similar in form and function to a threaded nut. The second member 132 may include an external thread or threaded region 138. In general, the external thread 138 is configured to engage the helical groove 136 of the first member 130. The second member 132 may also include an actuator 140. The second member 132 may also include a head or end surface 142 configured to engage the syringe plunger head 26. In this example, the end surface 142 may take the form of or otherwise include a relatively small projection or bump. This may allow for a smaller area of contact between the end surface 142 and the plunger head 26. This may help to reduce the likelihood that the plunger 24′ may rotate when engaging/interacting with the end surface 142 (e.g., which is rotating when the actuator 140 is rotated). Other devices disclosed herein may also include an end surface with a bump or projection. In a manner similar to what is described above in relation to FIGS. 3-4, the actuator 140 may be rotated. When doing so, the second member 132 may shift relative to the first member 130. The engagement between the end surface 142 and the syringe plunger head 26 may cause the plunger 24′ to shift within the syringe barrel 18.

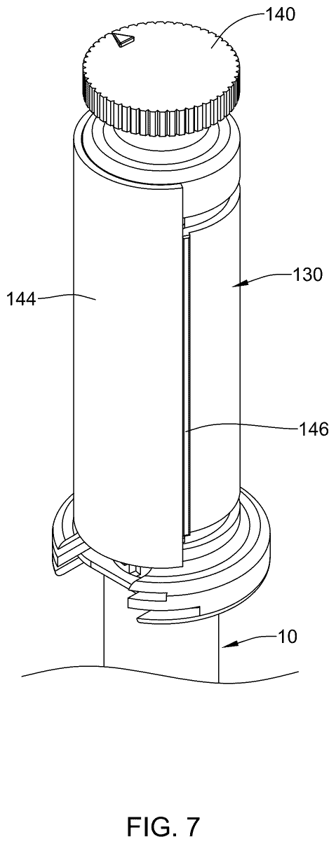

In some instances, the first member 130 may include an access door 144 that is configured to shift between an open position (e.g., as shown in FIG. 5) and a closed position (e.g., as shown in FIG. 7). When in the open position, a clinician may be able to insert the syringe 10 into the first member 130. When the syringe 10 is satisfactorily positioned, the access door 144 may be shifted toward the closed position. When in the closed position, the access door 144 may help to secure the syringe 10 within the first member 130. In some instances, the first member 130 may also include a stop member or region 146. The stop region 146 may help to stop the rotation of the access door 144 upon reaching the desired position and/or otherwise help to reduce the likelihood that the access door 144 is rotated further than desired. It can be appreciated that other devices disclosed herein may include an access door 144, as appropriate.

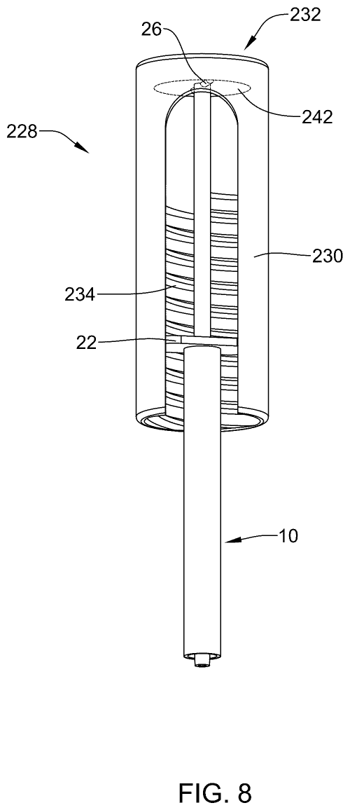

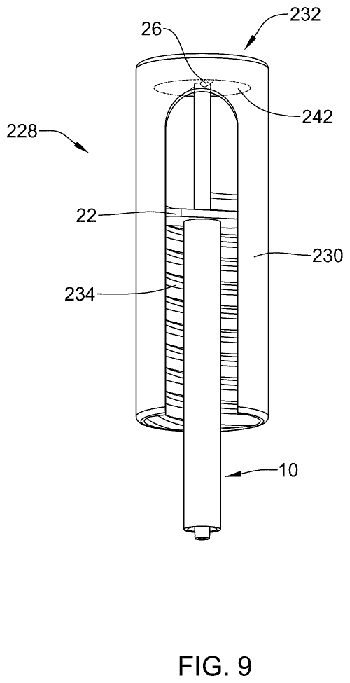

FIGS. 8-9 illustrate another example device 228 that may be similar in form and function to other devices disclosed herein. The device 228 may include a first member 230 and a second member 232 coupled to the first member 230. The first member 230 may include a holding region 234, which may take the form of a helical groove. In general, the holding region 234 may be configured to engage the flange 22 of the syringe 10. The second member 232 may include and/or take the form of an end surface 242 configured to engage the syringe plunger head 26. In at least some instances, the end surface 242 may be an end surface of the first member 130. Thus, the second member 232 may be defined at/by and end surface of the first member 130. In at least some instances, plunger head 26 may be axially fixed relative to the end surface 242.

In use, the first member 230 may be rotated relative to the syringe 10. When doing so, the flange 22 may shift within the first member 230 by translating along the holding region 234 (e.g., the helical groove). Because the syringe plunger head 26 may be axially fixed relative to the first member 230, rotation of the first member 230 relative to the syringe 10 may cause the plunger 24 to shift within the syringe barrel 18 as depicted in FIG. 9.

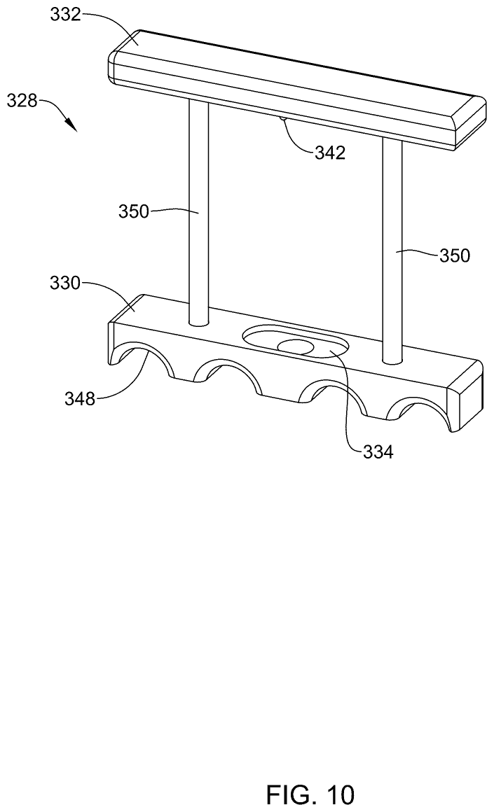

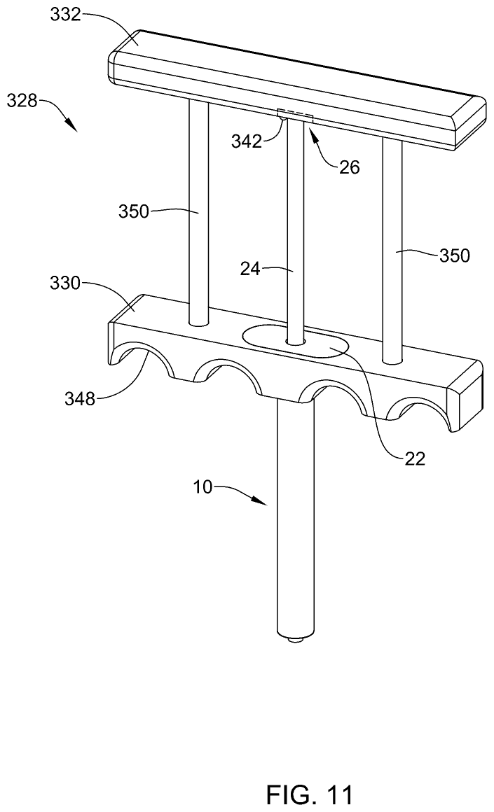

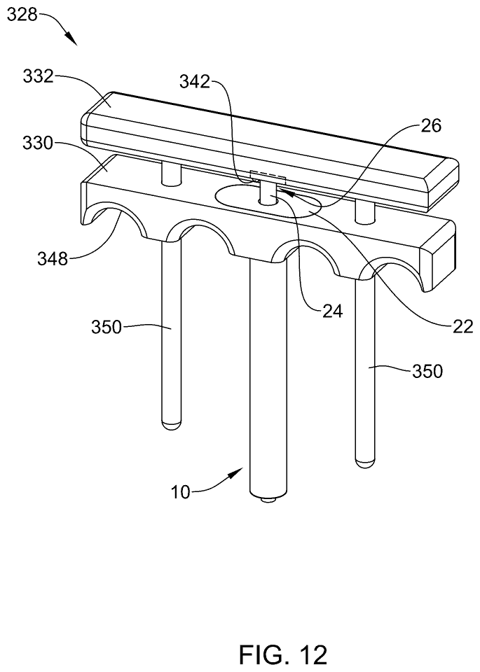

FIGS. 10-12 illustrate another example device 328 that may be similar in form and function to other devices disclosed herein. The device 328 may include a first member 330 and a second member 332 coupled to the first member 330. The first member 330 may include a holding region 334. In general, the holding region 334 may be configured to engage the flange 22 of the syringe 10 (see, for example, FIG. 11). For example, the holding region 334 may take the form of a cutout or recess that is configured to house the flange 22 therein. In some instances, the first member 330 may have a slot or cutout formed therein that allows a user to slide the flange 22 into the holding region 334 from the side. The first member 330 may include a grip region 348. The grip region 348 may have an ergonomic shape that allows a user to arrange their hand along the grip region 348 for a stable and comfortable grip. The second member 332 may include a head or end surface 342 configured to engage the syringe plunger head 26. In some instances, one or more stabilizing arms 350 may extend between the first member 330 and the second member 332. The stabilizing arms 350 may pass through openings in the first member 330. In use, the first member 330 and the second member 332 may be brought closer together. When doing so, the syringe plunger head 26 may cause the plunger 24 to shift within the syringe barrel 18 as depicted in FIG. 12.

The materials that can be used for the various components of the device 28 (and/or other devices disclosed herein) may include those commonly associated with medical devices. For example, the device 28 (and/or other devices disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A device for use with a syringe, the device comprising:
a first member having a holding region configured to engage a flange of a syringe barrel;
a second member coupled to the first member, the second member having an end surface configured to engage a syringe plunger head; and
wherein relative movement between the first member and the second member shifts the syringe plunger within the syringe barrel.

2. The device of claim 1, wherein the first member includes a substantially cylindrical body.

3. The device of claim 2, wherein the holding region includes a socket configured to secure the flange to the first member.

4. The device of claim 3, wherein a helical groove is formed along an interior region of the cylindrical body.

5. The device of claim 4, wherein the second member includes an actuator having an external thread, the external thread being configured to engage the helical groove.

6. The device of claim 5, wherein the second member is rotatable relative to the first member.

7. The device of claim 2, wherein the holding region includes a helical groove formed along an interior region of the cylindrical body.

8. The device of claim 7, wherein the cylindrical body is rotatable relative to the flange, and wherein rotation of the cylindrical body translates the flange relative to the syringe plunger.

9. The device of claim 1, wherein the first member includes a lower handle having one or more finger holding regions.

10. The device of claim 9, wherein the second member includes an upper push handle.

11. The device of claim 10, further comprising one or more alignment members extending between the first member and the second member.

12. An adapter for use with a syringe, the adapter comprising:
a substantially cylindrical body having a holding region configured to engage a flange of a syringe barrel;
a push member coupled to the substantially cylindrical body, the push member having an end surface configured to engage a syringe plunger; and
wherein relative movement between the substantially cylindrical body and the push member shifts the syringe plunger within the syringe barrel.

13. The adapter of claim 12, wherein the holding region includes a socket configured to secure the flange to the substantially cylindrical body.

14. The device of claim 13, wherein a helical groove is formed along an interior region of the substantially cylindrical body.

15. The device of claim 14, wherein the push member includes an external thread, the external thread being configured to engage the helical groove.

16. The device of claim 15, wherein the push member is rotatable relative to the substantially cylindrical body.

17. The device of claim 12, wherein the holding region includes a helical groove formed along an interior region of the cylindrical body.

18. The device of claim 17, wherein the substantially cylindrical body is rotatable relative to the flange, and wherein rotation of the substantially cylindrical body translates the flange relative to the syringe plunger.

19. An adapter for use with a syringe, the adapter comprising:
a lower handle having one or more finger regions and a socket configured to engage a flange of a syringe barrel;
an upper push handle coupled to the lower handle, the upper push handle having an end surface configured to engage a syringe plunger; and
wherein relative movement between the lower handle and the upper push handle shifts the syringe plunger within the syringe barrel.

20. The adapter of claim 19, further comprising one or more alignment members extending between the lower handle and the upper push member.
